(19) United States
(12) Patent Application Publication
(10) Pub. No.: US 2022/0273871 A1
(43) Pub. Date: Sep. 1, 2022

(54) INFUSION SYSTEM

(71) Applicant: Takeda Pharmaceutical Company Limited, Osaka-shi, Osaka (JP)

(72) Inventors: Dhairya Kiritkumar Mehta, Waltham, MA (US); Ling Zheng, Acton, MA (US)

(73) Assignee: Takeda Pharmaceutical Company Limited, Osaka-shi, Osaka (JP)

(21) Appl. No.: 17/626,414

(22) PCT Filed: Jul. 7, 2020
(86) PCT No.: PCT/US2020/040991

§ 371 Date: Jan. 11, 2022

Related U.S. Application Data
(60) Provisional application No. 62/873,684, filed on Jul. 12, 2019.

(51) Int. Cl. A61M 5/172; A61M 5/142; A61M 5/14

(52) U.S. Cl. CPC A61M 5/172; A61M 5/14228; A61M 5/1418; A61M 2205/8206; A61M 2205/502

(57) ABSTRACT

An infusion system includes a pump unit and a motor unit. The pump unit may be configured to receive and/or retain at least one container of medicinal fluid, and may include a fluid distribution system at least partially engaged with a peristaltic pump head disposed in the pump unit. The motor unit may be removably received in the pump unit and coupled to the peristaltic pump head to drive fluid from the container to a fluid outlet that is couplable to an infusion set.

### CROSS-REFERENCE TO RELATED APPLICATIONS

This application claims the benefit under 35 U.S.C. § 119(e) of U.S. Provisional Application No. 62/873,684, filed on Jul. 12, 2019, which is incorporated herein by reference in its entirety.

### FIELD

Disclosed embodiments are related to infusion systems and related methods of use.

### BACKGROUND

In some cases, conventional peristaltic infusion pumps may be employed to deliver medicinal fluids to a patient. These conventional peristaltic infusions pumps are large systems employed in hospitals that are typically connected to an intravenous bag containing medicinal fluid. Tubing in fluid communication with the intravenous bag is manually routed or coupled to a peristaltic pump head by a medical professional, after which the tubing may be connected to a patient so that the medicinal fluid may be delivered to the patient.

In other cases, peristaltic pumps may be employed in reusable reservoir based infusion systems which are worn by a patient. These systems typically include a refillable, integrated reservoir from which measured doses are delivered over time by the peristaltic pump. Such systems are completely integrated, and are periodically refilled.

### SUMMARY

In some embodiments, systems and methods for administering medicinal fluids to a patient with an infusion system having a pump unit and a motor unit are provided. In some embodiments, a pump unit includes a cavity to receive a container of medicinal fluid, a spike disposed in the cavity configured to pierce the container, a peristaltic pump head configured to pump fluid from the container, and a fluid outlet which is connectable to an infusion set. In some embodiments, a motor unit includes a motor unit housing containing a motor and a battery. In some embodiments, the motor unit may be received by the pump unit so that the motor is coupled to the peristaltic pump head. In some embodiments, the pump unit is disposable while the motor unit is reused with multiple pump units. In some embodiments, the pump unit and motor unit may be configured to be wearable on a patient. In some embodiments, a pump unit may be combined with a container for form a pump unit cartridge, where the container is retained in the pump unit and at least one blocking projection inhibits the container from moving in the pump unit to be pierced by the spike. The at least one blocking projection may inhibit movement of the container until a threshold force is applied to the container.

In some embodiments, a pump unit for an infusion system includes a housing and a cavity formed in the housing and configured to receive and support a container, where the cavity includes a cavity bottom and a cavity wall. The pump unit also includes a spike disposed in the cavity and extending perpendicular to the cavity bottom configured to pierce the container when the container is received in the cavity, a peristaltic pump head disposed in the housing, a fluid outlet, and tubing fluidly coupling the spike to the fluid outlet. The peristaltic pump head is in contact with at least a portion of the tubing, and the housing circumscribes the cavity, spike, peristaltic pump head, and tubing.

In some embodiments, a pump unit cartridge includes a housing, a cavity formed in the housing including a cavity bottom, and a cavity wall, and a container disposed in the cavity and including an internal cavity and a stopper, where the container is retained in the cavity and at least partially surrounded by the cavity wall. The pump unit cartridge also includes at least one blocking projection that inhibits the container from moving toward the cavity bottom, a spike disposed in the cavity and extending perpendicular to the cavity bottom configured to pierce the container when the container is moved toward the cavity bottom, a peristaltic pump head disposed in the housing, a fluid outlet, and tubing fluidly coupling the spike to the fluid outlet. The peristaltic pump head is in contact with at least a portion of the tubing.

In some embodiments, an infusion system includes a pump unit having a pump unit housing and a first cavity formed in the pump unit housing and configured to receive and support a container, where the first cavity includes a cavity bottom and a cavity wall. The pump unit also includes a first spike disposed in the cavity and extending perpendicular to the cavity bottom configured to pierce the container when the container is received in the cavity, a motor unit receptacle formed in the pump unit housing, a peristaltic pump head disposed in the housing, a fluid outlet, and tubing fluidly coupling the first spike to the fluid outlet, where the peristaltic pump head is in contact with at least a portion of the tubing. The infusion system also includes a motor unit disposed in the motor unit receptacle having a battery and a motor electrically connected to the battery and having an output shaft directly coupled to the peristaltic pump head. The motor unit is removable from the motor unit receptacle.

It should be appreciated that the foregoing concepts, and additional concepts discussed below, may be arranged in any suitable combination, as the present disclosure is not limited in this respect. Further, other advantages and novel features of the present disclosure will become apparent from the following detailed description of various non-limiting embodiments when considered in conjunction with the accompanying figures.

### BRIEF DESCRIPTION OF DRAWINGS

The accompanying drawings are not intended to be drawn to scale. In the drawings, each identical or nearly identical component that is illustrated in various figures may be represented by a like numeral. For purposes of clarity, not every component may be labeled in every drawing. In the drawings:

FIG. 1 is a front perspective view of one embodiment of an infusion system;

FIG. 2 is a rear perspective view of the infusion system of FIG. 1;

FIG. 3 is a first exploded view of the infusion system of FIG. 1;

FIG. 4 is a second exploded view of the infusion system of FIG. 1;

FIG. 5 is a front view of the infusion system of FIG. 1;

FIG. 6 is a side schematic of one embodiment of a motor and peristaltic pump head;

FIG. 7 is a schematic of a side view of one embodiment of an infusion system;

FIG. 8 is a schematic of a side view of one embodiment of an infusion system;

FIG. 9A is a schematic of a side view of one embodiment of a container and a cavity of an infusion system in a first position;

FIG. 9B is a schematic of a side view of the container and cavity of FIG. 9A in a second position;

FIG. 10A is a schematic of a side view of another embodiment of a container and a cavity of an infusion system in a first position;

FIG. 10B is a schematic of a side view of the container and cavity of FIG. 10A in a second position;

FIG. 11A is a schematic of a side view of another embodiment of a container and a cavity of an infusion system in a first position;

FIG. 11B is a schematic of a side view of the container and cavity of FIG. 11A in a second position;

FIG. 12 is a schematic of a side view of one embodiment of a spike;

FIG. 13 is a schematic of a top view of one embodiment of a pump unit;

FIG. 14 is a schematic of a side view of the pump unit of FIG. 13;

FIG. 15 is a schematic of a side view of the pump unit of FIG. 13 in use with one embodiment of a motor unit and containers;

FIG. 16 is a schematic of one embodiment of a fluid distribution system of a pump unit;

FIG. 17 is a schematic of one embodiment of an infusion set; and

FIG. 18 is a schematic of a side view of another embodiment of an infusion system.

### DETAILED DESCRIPTION

In some embodiments described herein, an infusion pump system includes a peristaltic pump unit. The inventors have recognized the benefits of a peristaltic pump unit for the delivery of medicinal fluids having large molecules. The inventors have recognized that a peristaltic pump, which does not come into fluid contact with medicinal fluid, may allow a medicinal fluid to be delivered to a patient with less drug particulate formation than other pumping methods. Peristaltic pump units of exemplary embodiments described herein may also have the benefits of easier setup and use for the infusion of a fluid, such that a peristaltic pumping unit may be operated at home or otherwise away from a professional medical facility.

Some conventional infusion pump systems are large, complex, and expensive machines that can be difficult to use. Many conventional infusion pumps are non-portable and fluidly couple to a medicinal fluid container or intravenous bag. Accordingly, these conventional infusion pumps may require regular sterilization for use, which can be a time consuming process. Additionally, many conventional infusion pumps are operated with some disposable parts, but these parts are oftentimes delivered to the patient as many separate components which may need to be assembled before use.

In view of the above, the inventors have also recognized the benefits of an infusion system which employs a modular arrangement including a motor unit and a pump unit. The motor unit may include durable, reusable parts such as a motor, battery, circuit board, communications devices, etc. The pump unit may be disposable and include a fluid distribution system (such as a spike, air inlet, and fluid outlet) and a peristaltic pump head. The pump unit may receive the motor unit or vice versa so that the durable components of the motor unit (for example, the motor) may connect to the peristaltic pump head so that fluid may be driven through the fluid distribution system. In some embodiments, the motor unit does not come into fluid contact with the medicinal fluid, so that the motor unit may be reused multiple times with different pump units. Each of the pump units may be disposed of after each use. The pump unit may be integrated into a housing, so that the setup of the infusion system may be as simple as positioning the motor unit into a motor unit receptacle formed in the pump unit. Such an arrangement may allow for easy setup of the infusion system, as the motor unit may be automatically aligned and coupled to the pump unit as the motor unit is received in the motor unit receptacle. Additionally, in contrast to conventional peristaltic pumping systems, as the fluid distribution system and peristaltic pump head may both be disposed in the pump unit, any peristaltic rollers or fingers may be pre-aligned with the fluid distribution system so that no further alignment is needed by a user. In some embodiments, a pump unit may include a cavity having a spike configured to receive and pierce a container of medicinal fluid, such that preparing the pump unit for an infusion process is as simple as inserting a vial into the cavity. In addition to the above, the infusion system of exemplary embodiments described herein may be sized and shaped to be worn on clothing of a patient without significantly encumbering the patient.

In conventional infusion systems, medicinal fluids are typically stored in separate containers or vials, or are stored in reusable refillable reservoirs contained inside of the infusion pump. In the case of medicinal fluids stored in separate vials, the containers are oftentimes manually pierced or opened so that the fluid may ultimately be transferred to an infusion set and delivered to a patient.

In view of the above, the inventors have also recognized the benefits of an infusion system including a pump unit cartridge having both a fluid distribution system and an untapped container of medicinal fluid integrated into the pump unit cartridge. The medicinal fluid container may be movably retained in a housing of the pump unit cartridge, so that when an infusion process is performed a user may apply a force to the container to pierce the container without needing to handle a separate container. In some embodiments, one or more blocking projections may inhibit the container from being pierced until a threshold force is applied to the container to avoid inadvertent piercing of the container. Such an arrangement may simplify the delivery of a medicinal fluid to a patient.

In some embodiments, a pump unit for an infusion system includes a housing with a cavity and a motor unit receptacle formed therein. In one embodiment, the cavity and motor unit receptacle may be formed on one side of the housing (e.g., a top side) opposite a pump unit bottom. A housing wall may circumscribe the cavity and the motor unit receptacle. Accordingly, in some embodiments, the pump unit may serve as a base into which a container of medicinal fluid and a motor unit may be placed by a medical professional or patient. The cavity may be sized and shaped to align the container relative to the pump unit as the container is received. Likewise, the motor unit receptacle may also align the motor unit relative to the pump unit as the motor unit is received in the motor unit receptacle. The pump unit may also include a fluid distribution system and a peristaltic pump head configured to move fluid through the fluid distribution system. In some embodiments, the peristaltic pump head may be part of a rotary peristaltic pump that includes a plurality of rotary rollers (e.g., three rollers) which sequentially contact a portion of the fluid distribution system to drive fluid through the fluid distribution system. In other embodiments, the peristaltic pump head may be part of a linear peristaltic pump that includes a plurality of compression elements arranged in a row which translate along parallel axes to sequentially contact a portion of the fluid distribution system and drive fluid through the fluid distribution system. In either embodiment, the peristaltic pump head may include an input shaft configured to couple to an output shaft of a motor unit which drives the peristaltic pump head. The housing wall of the pump unit may circumscribe the fluid distribution system and peristaltic pump head, so that the pump unit is self-contained.

In some embodiments, a motor unit for an infusion system includes a motor unit housing sized and shaped to be received in a motor receptacle of a pump unit. In one embodiment, the motor unit may include a motor having and output shaft and a battery electrically connected to the motor. The motor may be a DC motor, brushless motor, servomotor, or any other suitable electrical actuator. When the motor unit is coupled to a pump unit, the output shaft may couple to an input shaft of a peristaltic pump head of the pump unit. For example, one of the output shaft and input shaft may include a press fit coupling configured to receive the other with a suitable friction fit so that torque may be transmitted between the shafts. Of course, in other embodiments, any suitable coupling may be employed, such as magnetic couplings or other quick-connect or detachable couplings. In some embodiments, the motor unit may include a controller (e.g., a printed circuit board assembly with a processor configured to execute instructions stored in volatile or non-volatile memory) configured to control the activation and speed of the motor. According to this embodiment, the motor unit may include a user interface disposed on the motor unit housing which includes a screen which conveys information to a user and one or more buttons or other input devices. In some embodiments, the motor unit may include a communication device (e.g., a radio transceiver transmitting and receiving radio signals using one or more of Bluetooth, Bluetooth Low-Energy, Wi-Fi, 802.15.4, ZigBee, GSM, HSPA, CDMA, and/or any other suitable protocol) which communicates with a remote device such as a mobile phone or personal computer. According to this embodiment, a user may control and/or monitor the motor unit with the remote device.

It should be noted that, while exemplary embodiments are described herein with a pump unit receiving a motor unit and an input shaft receiving an output shaft, any suitable coupling between a motor unit and a pump unit may be employed, as the present disclosure is not limited in this regard. For example, in one embodiment, the motor unit may include a pump unit receptacle configured to receive the pump unit. In some embodiments, an output shaft of a motor unit may receive an input shaft of a pump unit. In one embodiment, a pump unit and motor unit may each include one receptacle and a corresponding portion of the housing which fits in the corresponding receptacle such that the pump unit and motor unit interlock.

In some embodiments, an infusion set for an infusion system includes a fluid connector, tubing, and a needle set. The fluid connector may be in fluid communication with the tubing and is configured to fluidly connect the infusion set to a fluid outlet of a pump unit. In some embodiments, the fluid connector may be a luer lock connector. The needle set may include at least one needle (e.g., one needle, two needles, three needles, four needles, etc.) configured to be inserted into a patient's body. In some embodiments, each of the needles may be configured as a butterfly needle. Of course, any suitable infusion needle may be employed, as the present disclosure is not so limited.

According to exemplary embodiments described herein, one or more components of an infusion system may be single-use disposables. In one embodiment, a pump unit and an infusion set may be single-use disposables, whereas a motor unit may be reusable. All of the medicinal fluid contacting components may be disposed in the disposable pump unit and infusion set, making the setup and use of a pump easier for a user.

In some embodiments, a pump unit may be combined with an integrated container of medicinal fluid to form a pump unit cartridge which allows a user to easily set up a pump unit for an infusion process. According to one embodiment, the container of medicinal fluid may be disposed in a cavity formed in the housing of the pump unit and retained therein. The container may be movable (e.g., slidable) in the pump unit between a first position and a second position. A spike or other piercing element disposed in the cavity may be configured to pierce the container as the container is moved from the first position to the second position to bring the container into fluid communication with a fluid distribution system of the pump unit. In some embodiments, at least one blocking projection formed on the pump unit or container may inhibit the container from being moved to the second position to avoid inadvertent piercing of the container. In one embodiment, application of a threshold force to the container may allow the container to be moved to the second position and pierced.

In some embodiments, a pump unit and/or motor unit may include a clip configured to allow the pump unit and/or motor unit to be worn on clothing. For example, in one embodiment, the clip may be configured as a belt clip formed on a pump unit housing which releasably attaches to a patient's belt. In another embodiment, the clip may be formed as a carabiner or spring latch configured to attach to a belt loop on a patient's pants. Of course, any suitable arrangement may be employed to allow a patient to wear the pump unit and/or motor unit, as the present disclosure is not so limited.

Turning to the figures, specific non-limiting embodiments are described in further detail. It should be understood that the various systems, components, features, and methods described relative to these embodiments may be used either individually and/or in any desired combination as the disclosure is not limited to only the specific embodiments described herein.

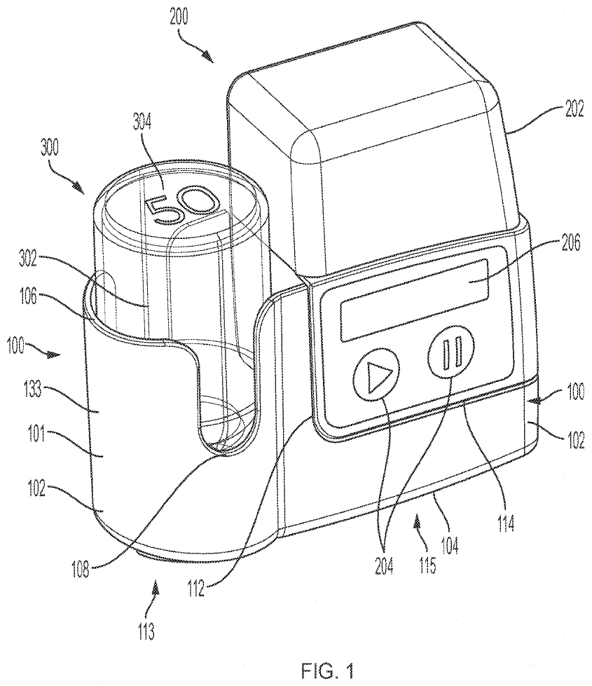

FIG. 1 is a front perspective view of one embodiment of an infusion system including a pump unit 100, a motor unit 200, and a container 300. According to the state shown in FIG. 1, the pump unit, motor unit, and container unit are all coupled to one another to form an infusion system, which may be coupled to an infusion set to deliver medicinal fluid from the container 300 to a patient.

As shown in FIG. 1, the pump unit 100 includes a pump unit housing 101 formed of a housing wall 102 and a housing bottom 104. The housing wall defines a perimeter of the pump unit and circumscribes other components of the pump unit. The pump unit housing defines a cavity 106 configured to receive and align the container 300 and a motor unit receptacle 110 configured to receive and align the motor unit 200.

In one embodiment as shown in FIG. 1, the cavity 106 includes at least one container slot 108, which extends from a top portion of the housing wall 102 toward the housing bottom 104. The container slot allows a user to see the container disposed in the cavity so that a user may determine a fluid level inside of the container. Such an arrangement may allow a user to determine if there is an occlusion or otherwise verify an infusion process is proceeding normally as fluid is drained from the container. Of course, while a slot is shown in FIG. 1, any suitable window or opening may be employed to allow a user to see the container disposed in the cavity, as the present disclosure is not so limited. In some embodiments, the housing wall may be formed of a transparent or semi-transparent material so that the container and fluid level therein may be viewed.

In some embodiments, the pump unit housing 101 includes a cuboid portion 115 and a cylindrical portion 113 having a circumferential wall 133. The cylindrical portion 113 may define the cavity 106, and the cuboid portion may define an area of the pump unit housing 101 that receives the motor unit 200. In some embodiments, the cylindrical portion 113 is longer than the cuboid portion 115 in a vertical direction.

According to the embodiment shown in FIG. 1, the motor unit receptacle 110 receives and supports at least two sides of the motor unit 200. More specifically, in the embodiment of FIG. 1, the motor unit receptacle includes at least a vertical portion 112 and a horizontal portion 114 which correspondingly support a motor unit side and a motor unit bottom, respectively. The vertical portion 112 may include two edges and the horizontal portion 114 may include three edges, so that the motor unit is supported by at least five edges of the motor unit receptacle. Such an arrangement may provide a more rigid connection between the motor unit and the pump unit for the transmission of torque between an output shaft of the motor unit and an input shaft of the pump unit. Of course, any suitable number of sides of the motor unit may be supported by the pump unit, including, but not limited to, one, two, three, and four sides.

As shown in FIG. 1, the motor unit 200 includes a motor unit housing 202 which houses the various components of the motor unit and is at least partially received in the motor unit receptacle 110. In the embodiment shown in FIG. 1, the motor unit housing 202 is arranged in a cuboid shape, although in other embodiments other shapes may be employed. Additionally, as noted previously, while in the embodiment of FIG. 1 the pump unit housing 101 receives the motor unit housing 202, in other embodiments the motor unit housing may receive the pump unit housing. In still other embodiments, the motor unit housing may both receive the pump unit (e.g., in a pump unit receptacle) and be received in the pump unit (e.g., in a motor unit receptacle), as the present disclosure is not so limited. The motor unit of FIG. 1 also includes a user interface composed of buttons 204 and a display screen. The buttons 204 may be used to control the functionality of the motor unit to correspondingly start, modify, or end and infusion process, among other functions. The buttons may be configured as mechanical switches, membrane switches, capacitive buttons, and/or any other suitable input device. The display screen may display information regarding the motor unit or an infusion process to a user. The display screen may be configured as an LCD screen, LED screen, E-Ink screen, OLED screen, or any other suitable display device.

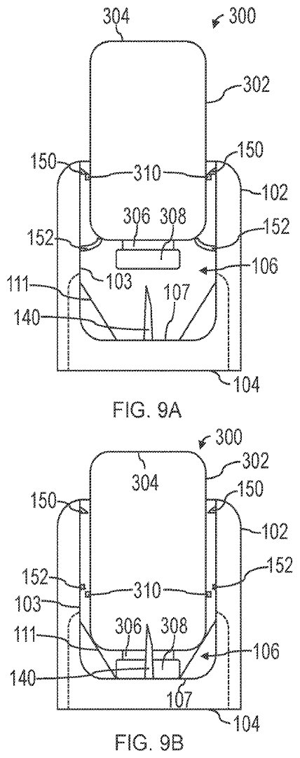

According to the embodiment of FIG. 1, the container 300 employed with the pump unit 100 may be configured as a vial having a container wall 302 and a container bottom 304 which define an internal volume in which a medicinal fluid containing a drug is disposed. According to the embodiment of FIG. 1, the container may be composed of glass, although other suitable materials may be employed such as plastic, as the present disclosure is not so limited. The container wall and/or container bottom may be transparent so that the fluid inside the container is visible to a user. The container may include an opening opposite the container bottom 304 which is sealed with a stopper. The stopper may be formed of rubber, silicone, or another material that may be pierced or otherwise broken with a spike or other tapping element disposed in the cavity 106. According to the embodiment of FIG. 1, the cavity 106 is configured to receive and align a plurality of differently sized containers 300. For example, while a 50 mL container is shown in FIG. 1, any suitably sized container may be employed, including, but not limited to containers having a volume greater than or equal to 1.25 mL, 2.5 mL, 5 mL, 10 mL, 20 mL, 30 mL, and 40 mL, 50 mL, 75 mL, 100 mL, 200 mL, and 300 mL. Examples of a container and its functionality with a cavity are described further with reference to the exemplary embodiments shown in FIGS. 9A-11B.

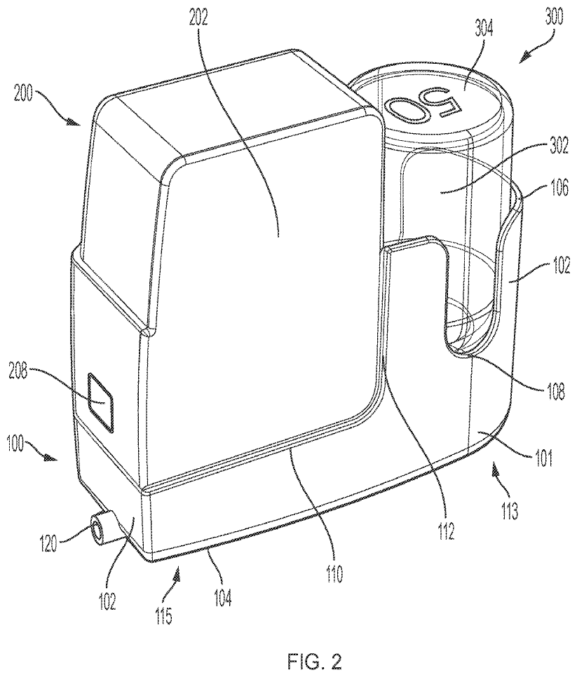

FIG. 2 is a rear perspective view of the infusion system of FIG. 1. According to the embodiment shown in FIG. 2, the pump unit 100 includes a second container slot 108 which extends from a top portion of the pump unit housing 101 toward the housing bottom 104 and allows a user to view a fluid level of the container 300 disposed in the cavity 106. Of course, while the pump unit of FIG. 2 includes two container slots, any suitable number of container slots, windows, or openings may be employed, as the present disclosure is not so limited. As shown in FIG. 2, the pump unit includes a fluid outlet 120 which is a part of a fluid distribution system disposed in the pump unit housing. In some embodiments, a fluid distribution system includes a fluid outlet, tubing, at least one spike, and an air inlet. The fluid outlet may be configured as a luer lock or other suitable fluid connector for connecting an infusion set.

According to the embodiment shown in FIG. 2, the motor unit 200 includes a port 208 which may be used to recharge a battery disposed in the motor unit housing 202. That is, the port 208 may receive a cable (such as a DC power cable, USB cable, or other suitable cable) which provides power from an external source to recharge an internal battery of the motor unit. Accordingly, in this embodiment, the motor unit may be operated wirelessly so that a patient is not encumbered or tethered to an external power source and may remain mobile. In other embodiments, a power cable may be connected to the motor unit via port 208 to power the motor unit directly from an external power source. In some embodiments, the port 208 may also be used to pass information to an external device such as a mobile phone or personal computer. Such a port may be employed to allow one or more parameters of the motor unit to be configured or to download diagnostic or usage data from the motor unit, among other uses.

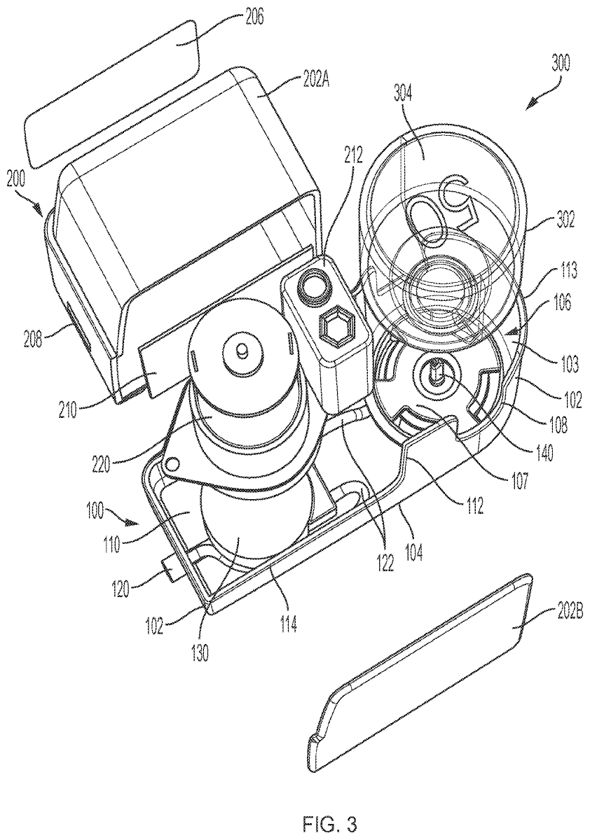

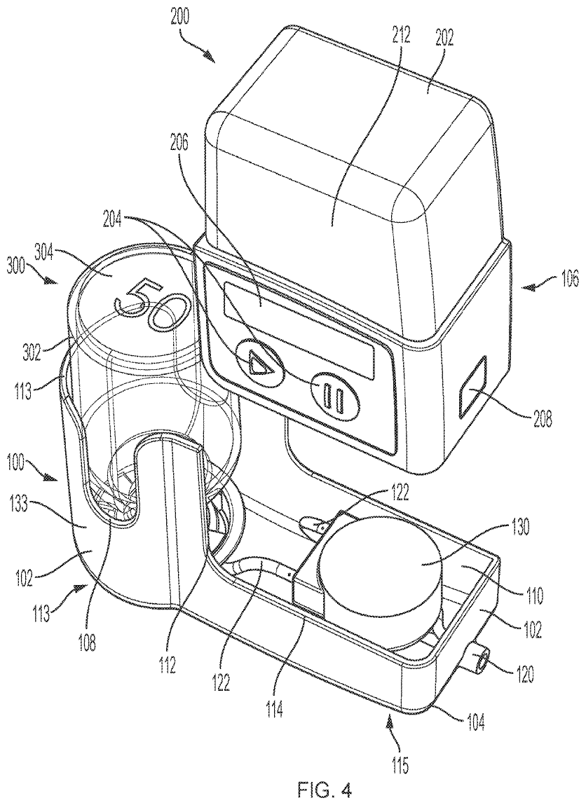

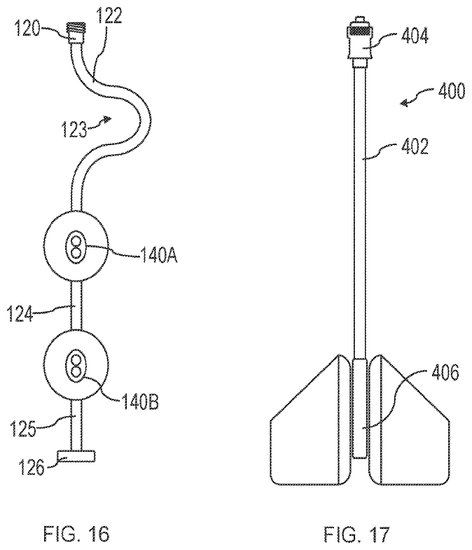

FIGS. 3 and 4 are first and second exploded views, respectively, of the infusion system of FIG. 1. As shown in FIG. 3 and discussed above, the pump unit of FIG. 3 houses a fluid distribution system which moves fluid from the container 300 to the fluid outlet 120. The fluid distribution in the depicted embodiment includes tubing 122 and a spike 140. The tubing fluidly connects the spike to the fluid outlet 120 and a portion of the tubing is engaged with a peristaltic pump head 130 which is also disposed inside of the pump unit housing 101. The spike 140 is configured to pierce a stopper of the container 300 and fluidly connect an internal, fluid filled lumen of the container to the fluid distribution system. Additionally, the fluid distribution includes an air inlet (for example, see FIG. 16) which is fluidly connected to the spike and allows air into a connected container to inhibit vacuum formation which may prevent or slow fluid flow from the container. According to the embodiment shown in FIGS. 3-4, the peristaltic pump head 130 is a rotary peristaltic pump head and includes a plurality of rollers which sequentially engage a portion of the tubing 122 to advance fluid disposed in the tubing toward the fluid outlet. In some embodiments, the peristaltic pump head may advance an individual bolus of fluid, while in other embodiments the peristaltic pump head may advance fluid continuously. According to the embodiment shown in FIGS. 3-4, the housing wall 102 circumscribes the tubing 122, peristaltic pump head 130, and spike 140. In addition, the tubing 122, peristaltic pump head 130, and spike 140 are disposed below an uppermost portion 13 of the pump unit 100.

As shown in FIG. 3, the motor unit housing is configured in a first section 202A and second section 202B which are configured to be secured together around the internal components of the motor unit 200. In the depicted embodiment, the motor unit includes a controller circuit board 210 (e.g., a printed circuit board assembly), a battery 212, and a motor 220. The battery is electrically connected to the motor and/or circuit board 210, which in turn controls the delivery of electrical power from the battery to the motor 220. The motor 220 shown in FIG. 3 is a DC motor, although other motor types may be employed. The controller circuit board 210 controls the on or off state and speed of the motor to corresponding control the speed of the peristaltic pump head 130 to ultimately control the rate of flow of a fluid through the fluid distribution system of the pump unit 100. The circuit board 210 is also electrically connected to a screen 206 which relays information about the motor unit (such as operational state, pumping speed, etc.) to a user of the infusion system.

According to the embodiment shown in FIG. 3, the cavity 106 includes a cavity wall 103 and a cavity bottom 107. The cavity wall and cavity bottom define a cavity volume sized and shaped to receive the container 300. The spike 140 is disposed on the cavity bottom and projects perpendicularly from the cavity bottom such that the spike is oriented along a longitudinal axis of the cavity volume. In the embodiment of FIG. 3, the cavity wall is configured such that the container 300 is aligned and guided by the cavity wall as the container is moved toward the cavity bottom. In particular, at least a portion of the container wall 302 contacts at least a portion of the cavity wall 103 to orient the container relative to the spike 140. Such an arrangement ensures the spike is aligned with the container to improve the ease of spiking the container.

FIG. 4 depicts the assembled motor unit 200 of FIG. 1 decoupled from the pump unit 100. As shown in FIG. 4 and discussed previously, the motor unit receptacle 110 includes a vertical portion 112 and a horizontal portion 114 which receive corresponding portion of the motor unit housing 202. That is, the motor unit housing has a size and shape corresponding to that of the motor unit receptacle so that the motor unit is reliably received and supported in the motor unit receptacle. Additionally, in the embodiment of FIG. 4, the motor unit receptacle ensures the motor unit is aligned with the peristaltic pump head 130 to achieve a reliable mechanical connection between the motor of the motor unit and the peristaltic pump head.

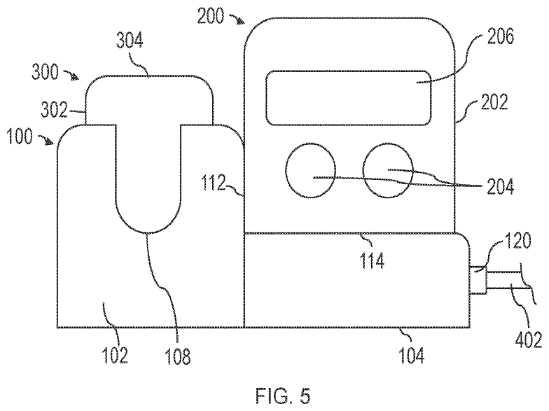

FIG. 5 is a front schematic view of the infusion system of FIG. 1. As shown in FIG. 5 and discussed previously, the pump unit includes a housing wall 102 which circumscribes the components of the pump unit. In the embodiment of FIG. 5, a container slot 108 is formed in the housing wall and allows a user to see a majority of the container 300 while the housing wall still extends for along a majority of a longitudinal length of the container. Additionally, a motor unit receptacle includes a vertical portion 112 and a horizontal portion 114 which support the motor unit 200 on two sides. As shown in FIG. 5, the fluid outlet 120 is coupled to infusion tubing 402 of an infusion set which may be fluidly connected to a needle set for infusion into a patient.

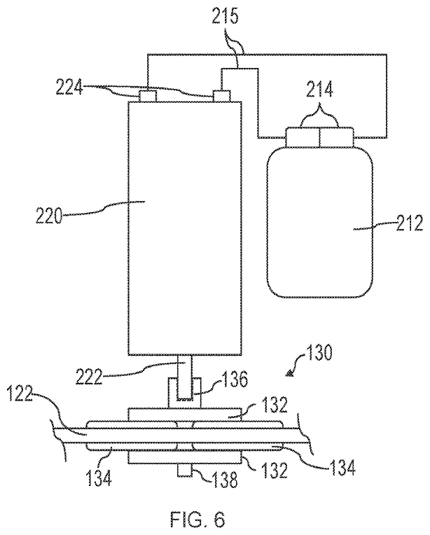

FIG. 6 is a side schematic of one embodiment of a motor 220 and peristaltic pump head 130. According to the embodiment of FIG. 6, the peristaltic pump head is configured as a three roller rotary peristaltic pump head. The peristaltic pump head includes a chassis 132 which carries a plurality of rollers 134. Each of the rollers is rotatably retained inside the housing, so that a roller engaged with tubing 122 of a pump unit is able to roll over the tubing. Such an arrangement may limit frictional wear on the tubing. The peristaltic pump head also includes an input shaft 136 and an axle 138 about which the chassis rotates. The axle may be retained in a pump unit housing such that the chassis is rotatably coupled to the chassis. According to the embodiment of FIG. 6, the input shaft 136 is configured to receive an output shaft 222 of the motor (for example, when a motor unit is received in a motor unit receptacle). In particular, in the embodiment of FIG. 6, the input shaft 136 receives the output shaft 222 of the motor in a press fit, mechanical interlock, or any other suitable arrangement so that torque may be transmitted between the output shaft and the input shaft. Accordingly, in the embodiment of FIG. 6, the output shaft is directly coupled to the peristaltic pump head.

As shown in FIG. 6, the motor 220 includes an output shaft and a pair of motor terminals 224. The motor of FIG. 6 is configured as a DC motor which receives a voltage at the motor terminals and generates a torque in the output shaft 222. The speed and torque of the motor may be controlled by the voltage supplied to the terminals, either via an analog voltage or through pulse width modulation (PWM). Accordingly, in some embodiments, a controller (e.g., a printed circuit board assembly having a processor executing instructions stored in volatile or non-volatile memory) may control the torque and speed of the motor. As shown in FIG. 6, the motor terminals 224 are connected via wires 215 to corresponding battery terminals 214 of a battery 212. The battery may be any suitable power source which provides electrical power to the motor 220 and/or a controller. For example, the battery 212 may be Li-ion, Li—Po, Ni—Cd, Ni-MH, or any other suitable battery. In some embodiments, the battery 212 may be rechargeable and reused for multiple infusion processes. In other embodiments, the battery may be replaceable and periodically replaced as multiple infusion processes are performed.

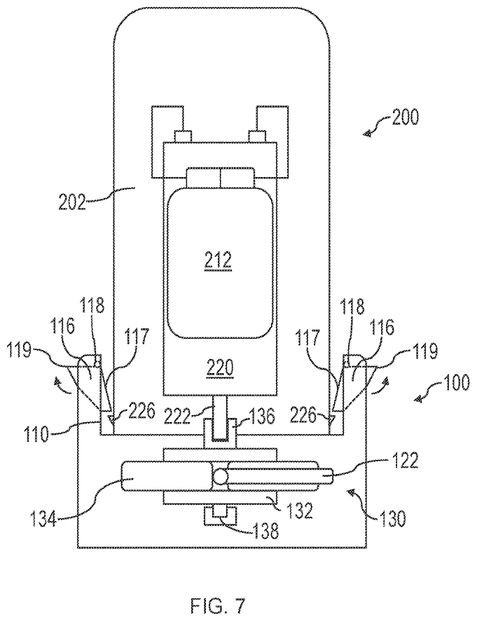

FIG. 7 is a side schematic of one embodiment of an infusion system showing an interface between a pump unit 100 and a motor unit 200. According to the embodiment of FIG. 7, the motor unit includes a motor 220 and a battery 212 similar to those shown in FIG. 6. The pump unit includes a peristaltic pump head 130 which is rotatably mounted in the pump unit and rotates about axle 138. Accordingly, in the configuration shown in FIG. 7, the motor 220 may transfer torque to the peristaltic pump head 130 via output shaft 222 coupled to input shaft 136. As noted previously, while the input shaft 136 receives the output shaft 222 in the embodiment of FIG. 7, in other embodiments the output shaft may receive the input shaft. According to the embodiment of FIG. 7, the corresponding shape of the motor unit housing 202 and motor unit receptacle 110 may automatically orient and align the output shaft and the input shaft for proper engagement as the motor unit is received in the pump unit.

As shown in FIG. 7, the pump unit 100 is configured to receive the motor unit 200 in a motor unit receptacle 110. The motor unit receptacle is sized and shaped to receive the motor unit housing 202. In the embodiment of FIG. 7, motor unit projections 226 are formed on an exterior of the motor unit housing and are configured to engage latches 116 of the pump unit. According to the embodiment of FIG. 7, the latches 116 are integrated into the motor unit receptacle and include an engagement projection 117, a hinge 118, and a lever 119. The latch is configured to rotate between an engaged position and a disengaged position about the hinge 118. The hinge may be a pin, living hinge, or any other suitable arrangement to allow rotation of the latch. In the engaged position, the engagement projection 117 protrudes into the motor unit receptacle 110 so that the engagement projection overlaps with a corresponding motor unit projection 226. Accordingly, in the engaged position, the latches inhibit the removal of the motor unit 200 from the motor unit receptacle to ensure any inadvertent contact (e.g., bumping) does not dislodge the motor unit from the motor unit receptacle. Additionally, the engagement projections may ensure the output shaft 222 stays engaged with the input shaft 136. According to the embodiment of FIG. 7, the levers 119 are user operable components that may be used to rotate the latches from the engaged position to the disengaged position. That is, the levers 119 may be used to rotate the latches in the direction shown by the arrow to move the engagement projections 117 out of alignment with the motor unit projections 226. Accordingly, in some embodiments, in the disengaged position, the latches do not inhibit the removal of the motor unit from the pump unit so that the motor unit may be removed and reused with other pump units.

According to the embodiment of FIG. 7, the latches 116 are configured to rotate in an over center arrangement, such that force applied to the motor unit housing 202 to remove the motor unit 200 from the motor unit receptacle 110 moves the latches into further engagement with the motor unit projections 226. Put another way, the force transmitted from the motor unit projections 226 to the engagement projection 117 of the latches generates a moment on the latches in a direction toward the engaged position rather than a disengaged position. Such an arrangement may promote secure engagement of the motor unit in the motor unit receptacle so that the motor unit may only be removed if a user depresses the levers 119. Of course, in other embodiments the latches may only resist the removal of the motor unit until a removal threshold force is reached, as the present disclosure is not so limited. In some embodiments, the latches 116 maybe biased toward the engaged position. For example, a torsion spring, compression spring, or tension spring may be employed to bias the latches toward the engaged position. As another example, the latch may be a resilient, flexible member which generates biasing force when deflected from a resting position. Of course, any suitable biasing arrangement may be employed, as the present disclosure is not so limited.

In the embodiment of FIG. 7, the latches 116 are configured to move to the disengaged position as the motor unit 200 is received in the motor unit receptacle 110. That is, the engagement projections 117 of the latches are shaped such that force applied to the motor unit in a direction of the motor unit receptacle generates a moment on the latches which rotates the latches in a direction toward the disengaged position (e.g., in the direction of the arrows). Such an arrangement allows the motor unit to be easily coupled to the pump unit 100 with a single force application without operating the levers 119. Of course, other embodiments may be employed where one or more of the latches 116 may be moved to the disengaged position prior to the motor unit being coupled to the motor unit receptacle, as the present disclosure is not so limited.

It should be noted that while two latches 116 and corresponding motor unit projections 226 are depicted in the embodiment of FIG. 7, any suitable number of latches and motor unit projections or latch receptacles may be employed, as the present disclosure is not so limited. For example, a single latch and corresponding motor unit projection or latch receptacle may be employed. It should also be noted that while in the embodiment of FIG. 7 the pump unit includes latches 116 and the motor unit 200 includes motor unit projections 226, in other embodiments the latches may be disposed on the motor unit and latch receptacles, or corresponding projections may be disposed on the pump unit. In still other embodiments, each of the pump unit and motor unit may include at least one latch and at least one corresponding latch receptacle or projection. In some embodiments, instead of being retained together with latches, a pump unit and motor unit may be retained together with a friction fit, snap fit, or any other suitable configuration.

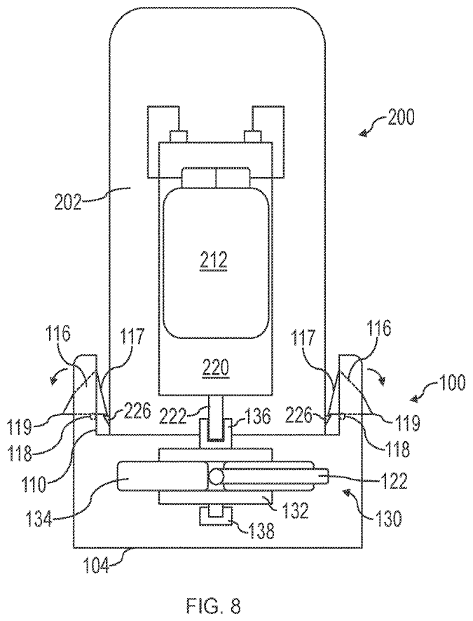

FIG. 8 is a side schematic of one embodiment of an infusion system showing an alternative latching arrangement between the pump unit 100 and the motor unit 200. Similar to the embodiment of FIG. 7, the pump unit includes two latches 116, each having an engagement projection 117, a hinge 118, and a lever 119. However, in contrast to the embodiment of FIG. 7, the hinge 118 of each latch is disposed on an opposite side of the latch. That is, the hinge is disposed on a housing bottom 104 side of the latch, such that the direction of rotation of the latch between engaged and disengaged positions is reversed relative to the embodiment of FIG. 7. Such an arrangement may ensure a close fit between the engagement projections 117 and the motor unit projections 226 while mitigating any interference or jamming between the latches and the motor unit housing. Moreover, the embodiment of FIG. 8 retains the motor unit in a motor unit receptacle 110 until a threshold force is applied to the motor unit in a direction of removal. As force is applied to remove the motor unit, the motor unit projections 226 apply a moment to the latches 116 about each hinge to rotate the latch toward the disengaged position. The latch 116 may be biased toward the engagement position to retain the motor unit in the housing until a removal threshold force is applied to the motor unit. This removal threshold may be tuned by the geometry and biasing force to any suitable value. Such an arrangement may allow a user to couple and decouple the motor unit 200 to the pump unit with simple force applications.

FIGS. 9A-11B depict various embodiments of a container and a container cavity formed in a pump unit. According to the exemplary embodiments described below, the container and pump unit may be integrated together as a pump unit cartridge. That is, the container may be retained in the cavity while remaining fluidly isolated from other components in the pump unit. According to the embodiments of FIGS. 9A-11B, the container and/or pump unit includes at least one blocking projection which inhibits the container from moving toward a spike or other fluid coupling so that the container remains fluidly isolated from the other components of the pump unit. A user may depress the container or otherwise activate the pump unit to bring the container into fluid communication with a fluid distribution system of the pump unit. Such an arrangement allows for simple setup for an infusion process.

FIG. 9A is a side schematic of one embodiment of a container 300 and a cavity 106 of an infusion system in a first position. As shown in FIG. 9A, the container is configured as a vial and includes a container wall 302 and a container bottom 304 which define an internal volume filled with medicinal fluid. The container also includes a neck 306 defining an opening which is sealed with a stopper 308. The stopper may be formed of rubber, silicone, or another suitable material. The container also includes container projections 310 formed on an exterior of the container wall 302. According to the embodiment of FIG. 9A, the cavity 106 includes a cavity wall 103 and a cavity bottom 107 which are sized and shaped to receive the container 300. A spike 140 is disposed in the cavity and projects perpendicularly from the cavity bottom so that that spike is aligned with a longitudinal axis of the cavity and the container 300. Accordingly, when the container is moved toward the cavity bottom 107, the spike 140 pierces the stopper 308, thereby bringing the internal volume of the container into fluid communication with a fluid distribution system of the pump unit.

According to the embodiment in FIG. 9A, the cavity includes a universal alignment portion 111, which in the present embodiment is arranged as an annular inclined surface directed toward the spike. The universal alignment portion is configured to receive, orient, and align a plurality of differently sized containers with the spike 140. For example, a container having a smaller diameter than that of FIG. 9A may contact the universal alignment portion and be moved into alignment with the spike, even if the cavity wall 103 is spaced from the smaller container. In this manner, a plurality of different sized containers may be employed with a single pump unit 100, which may increase simplicity and reduce cost in manufacturing. Of course, while an inclined surface is shown in FIG. 9A, any suitable universal alignment portion may be employed, including guides, biasing members, or other configurations. For example, in some embodiments, the universal alignment portions may move to accommodate larger containers. According to this embodiment, the universal alignment portions may be spring-biased toward a resting position where a smallest container is supported by the universal alignment portions. The universal alignment portions may move against the spring-bias force to accommodate and support a larger container.

As shown in FIG. 9A, the cavity 106 includes blocking projections in the form of container retainers 150 and frangible projections 152 which limit the movement of the container in the cavity. The container retainers engage the container projections 310 to inhibit removal of the container from the cavity. Accordingly, the container and pump unit 100 may function as an integrated unit which is delivered to a patient as one piece. The frangible projections are in contact with the container wall 302 and inhibit the movement of the container in a direction toward the cavity bottom 107. Put another way, the frangible projections support the weight of the container 300 and inhibit the movement of the stopper 308 toward the spike 140 so that the container remains unpierced. Accordingly, in the position shown in FIG. 9A, the container is fixed relative to the cavity 106, and the pump unit may be handled or shipped without the container being inadvertently pierced. The frangible projections 152 may resist movement of the container toward the cavity bottom 107 until a threshold force is applied to the container, whereupon the frangible projections may break to allow the container to be moved toward the cavity bottom and pierced by the spike, as shown in FIG. 9B. In this manner, a user preparing for an infusion process may apply a single threshold force to the container to prepare a pump unit for an infusion process without needing to place a container in a correct location.

FIG. 9B is a side schematic of the container 300 and cavity 106 of FIG. 9A in a second position. As shown in FIG. 9B, the container has been moved toward and into contact with the cavity bottom 107 relative to FIG. 9A. Correspondingly, the spike 140 has pierced the stopper 308 to bring the internal volume of the container into fluid communication with a fluid distribution system connected to the spike. Accordingly, fluid may be withdrawn from the container and delivered to a patient. As shown in FIG. 9B, the frangible projections have been broken off by the application of a threshold force to the container. For example, the threshold force may have been applied by a user providing a pushing force on the container bottom 304.

It should be noted that while two frangible projections 152 are shown in FIGS. 9A-9B, any suitable number of frangible projections may be employed to resist movement of the container 300 until a threshold force is applied. Additionally, while the frangible projections shown in FIGS. 9A-9B are shaped as fingers, any suitable shape of the frangible projections may be employed.

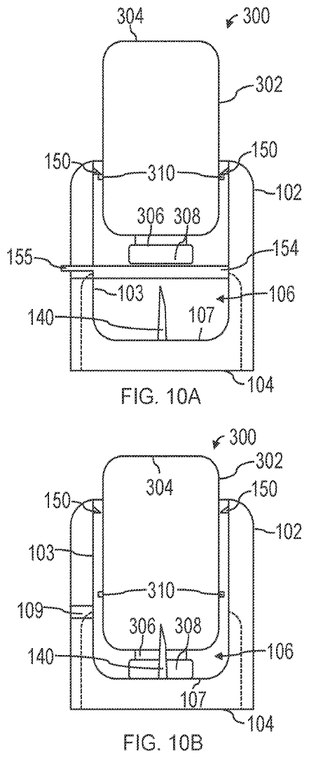

FIG. 10A is a side schematic of another embodiment of a container 300 and a cavity 106 of an infusion system in a first position. Like the embodiment of FIG. 9A-9B, the container 300 is retained in the cavity 106 via container retainers 150, which contact the container projections 310 and inhibit removal of the container. According to the embodiment of FIG. 10A, the cavity 106 includes a blocking projection in the form of blocking plate 154 which is disposed between a spike 140 in the cavity and a stopper 308 of the container. The blocking plate 154 inhibits movement of the container toward a cavity bottom 107. In this manner, the blocking plate maintains the position of the container relative to the cavity. In contrast to the embodiment of FIGS. 9A-9B, the blocking plate is not configured to be broken during use. Rather, the blocking plate includes a pull-tab 155 which may be pulled by a user to remove the blocking plate from the cavity. Accordingly, to pierce the container 300, the pull-tab 155 may be pulled and the container subsequently depressed to pierce the stopper 308 and bring the container into fluid communication with a fluid distribution system, as shown in FIG. 10B.

FIG. 10B is a side schematic of the container 300 and cavity 106 of FIG. 10A in a second position where the stopper 308 is pierced by the spike 140. As noted above, the blocking plate has been removed from the cavity. In the embodiment of FIG. 10B, the blocking plate may be removed via a pull-tab which is used to pull the blocking plate out of a blocking plate slot 109 so that the container is able to move to a lower or engaged position shown in FIG. 10B from the upper or disengaged position shown in FIG. 10A.

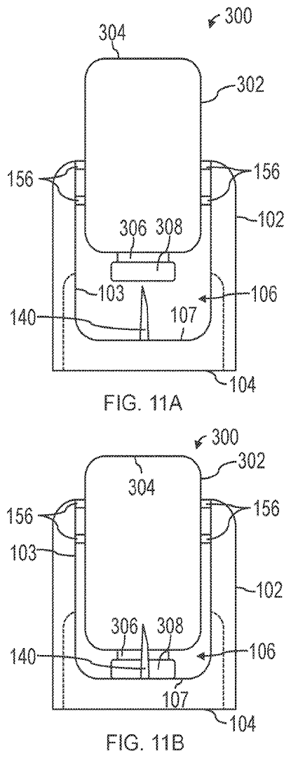

FIG. 11A is a side schematic of another embodiment of a container 300 and a cavity 106 of an infusion system in a first position. As shown in FIG. 11A, and similar to the embodiments of FIG. 9A-10B, the cavity includes a cavity wall 103 and a cavity bottom 107 which are sized and shaped to receive the container. A spike 140 projects from the cavity bottom and is configured to pierce a stopper 308 of the container when the container is moved from an upper or disengaged position shown in FIG. 11A to the lower or engaged position shown in FIG. 11B. However, in contrast to the prior embodiments, the container 300 is inhibited from being removed from the cavity or moved toward the cavity bottom up to a threshold force. Rather than having rigid projections, the cavity includes blocking projections in the form of compressible joints 156 disposed between and in contact with both the cavity wall 103 and the container wall 302. The compressible joints 156 are composed of a compressible material such as rubber, silicone, or another suitable material such that the joints 156 are compressed between the cavity wall and container wall. Accordingly, the compressible joints generate static and kinetic frictional forces between the container and the cavity wall which resist the motion of the container relative to the cavity. In some embodiments, the compressible joints may be arranged as O-rings which are disposed around a circumference of the cavity wall 103.

In the embodiment of FIG. 11A, the compressible joints are arranged such that the static frictional force is sufficient to inhibit movement of the container until a threshold force is applied to the container. For example, gravity, general jostling, or bumping may be forces insufficient to move the container relative to the cavity. Accordingly, the infusion system may be delivered to a user with the container attached in this manner to help avoid inadvertent piercing of the stopper 308. When setting up the infusion system for an infusion process, a threshold force may be applied to the container 300 (e.g., the container bottom 304) to move the container toward the cavity bottom to pierce the stopper 308 with the spike 140, as shown in FIG. 11B.

FIG. 11B is a side schematic of the container 300 and cavity 106 of FIG. 11A in a second position. As shown in FIG. 11B, the stopper 308 has been pierced by the spike 140 as the container is in a lower or engaged position. From the position shown in FIG. 11A, a threshold force was applied to the container to move the container against the resistive frictional forces of the compressible joints 156.

While two compressible joints 156 are shown in the embodiment of FIGS. 11A-11B, any suitable number of compressible joints may be employed to provide a desired resistive force to the movement of the container 300. For example, one compressible joint may be employed or three compressible joints may be employed. Furthermore, while annular compressible joints are discussed with reference to FIGS. 11A-11B, any suitable continuous or non-continuous shape of the compressible joints may be employed, and the compressible joints may contact any portion of the container wall 302 to provide resistive forces. For example, the compressible joints may be formed as a plurality of distinct patches of material that are spaced from one another. Additionally, the compressible joints may be primarily disposed on either the cavity wall 103 or container wall 302, as the present disclosure is not so limited.

It should be noted that the exemplary embodiments described with reference to FIGS. 9A-11B may be employed in any desirable combination. That is, frangible projections, blocking plates, and compressible joints may be employed individually, in partial combination, or full combination in a cavity, as the present disclosure is not so limited.

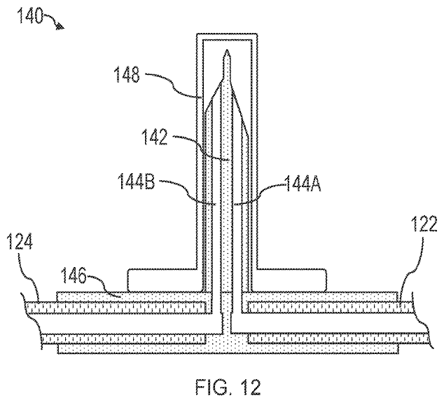

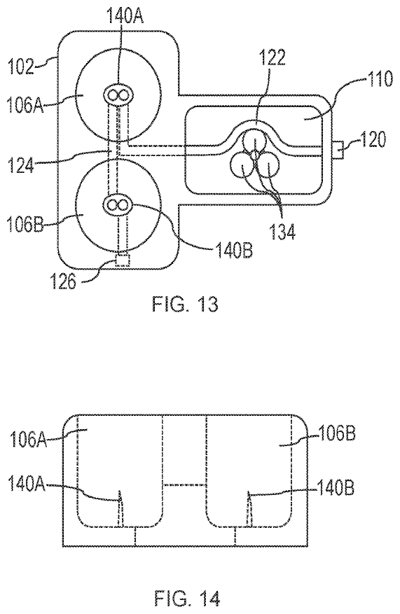

FIG. 12 is a side schematic of one embodiment of a spike 140 which may be employed in infusion systems of exemplary embodiments herein. As shown in in FIG. 12, the spike includes a spike body 142 which includes a first lumen 144A and a second lumen 144B. The spike body is coupled to a spike base 146 which receives first tubing 122 and second tubing 124 which continue fluid channels defined by the first lumen and second lumen, respectively. The spike body 142 is configured to pierce a stopper of a container and bring the first lumen and second lumen into fluid communication with the container. Once disposed in the container, fluid from the container may flow down the first lumen and/or second lumen under the effects of gravity or pumping (e.g., differential pressure). In the embodiment of FIG. 12, the second tubing 124 may coupled to an air inlet which allows air to be introduced into a container via the second lumen 144B to mitigate vacuum formation in the container. Accordingly, medicinal fluid may flow primarily down the first lumen 144A and ultimately be delivered to patient. In some embodiments, multiple spikes may be serially linked so that fluid may be delivered simultaneously from multiple containers, as will be discussed further with reference to FIGS. 13-15.

In one embodiment as shown in FIG. 12, the spike 140 may include a spike sheath 148 which surrounds the spike body 142. The spike sheath protects the spike 140 prior to a container being pushed onto the spike and pierced. As the container is advanced over the spike, the spike sheath may be broken so that the first and second lumens 144A, 144B may be brought into fluid communication with the container. In an embodiment where multiple spikes are serially linked, the spike sheaths may inhibit fluid leakage from the first and second lumens if the containers are sequentially pierced by the spikes.

FIG. 13 is a top schematic of one embodiment of a pump unit 100 including a first cavity 106A and a second cavity 106B which are each configured to receive a container of medicinal fluid. Each cavity includes a spike 140A, 140B, which are serially linked to one another. That is, a second spike 140B is coupled to an air inlet 126 on one end and a first spike 140A on the other end via second tubing 124. The air inlet may include a hydrophobic filter which allows air into a fluid distribution system formed partially by the spikes and second tubing without allowing fluid passage. Accordingly, the first spike and second spike are fluidly linked and are able to pool fluid from both containers which are received in the first cavity and second cavity. The first spike 140A is fluidly connected to a fluid outlet 120 via first tubing 122. A portion of the first tubing is engaged with a peristaltic pump head having three rotary rollers 134. The peristaltic pump head is disposed in a motor unit receptacle 110 so that an output shaft may be coupled to the peristaltic pump head to drive fluid from the received containers to the fluid outlet.

FIG. 14 is a side schematic of the pump unit 100 of FIG. 13. As shown in FIG. 14, the pump unit includes two distinct cavities 106A, 106B which are each sized and shaped to receive a container of medicinal fluid. Spikes 140A, 140B are disposed in the cavities and project perpendicularly from a bottom of each cavity.

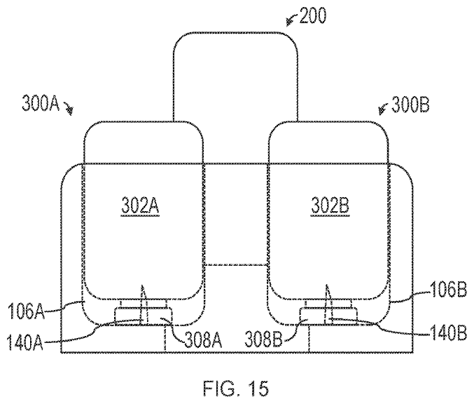

FIG. 15 is a side schematic of the pump unit 100 of FIG. 13 in use with one embodiment of a motor unit 200 and containers 300A, 300B. In FIG. 15, container walls 302A, 302B, of each container are configured to match the shape of the cavities 106A, 106B such that the containers are oriented and aligned with the spikes 140A, 140B when the containers are disposed in the cavities. As shown in FIG. 15, the spikes 140A, 140B, have pierced stoppers 308A, 308B of each container to bring both containers into fluid communication with the fluid distribution system of the pump unit to allow the total volume of medicinal fluid to be delivered from a fluid outlet of the pump unit.

FIG. 16 is a schematic of one embodiment of a fluid distribution system of a pump unit. As shown in FIG. 16, the fluid distribution system includes a fluid outlet 120, first tubing 122, a first spike 140A, second tubing 124, a second spike 140B, third tubing 125, and an air inlet 126. The fluid outlet 120 of FIG. 16 is configured as a female luer lock valve, which restricts fluid flow through the outlet until a corresponding male luer lock is connected to the fluid outlet. As noted previously, the air inlet 126 may include a hydrophobic filter configured to allow air into the fluid distribution system while preventing fluid passage. According to the embodiment of FIG. 16, the spikes 140A, 140B are configured as dual-lumen spikes connected in series similar to the spike shown in FIG. 12. Of course, in other embodiments, single-lumen or other multi-lumen spikes may be employed, as the present disclosure is not so limited. Additionally, it should be noted that any suitable number of spikes may be employed in a fluid distribution system to pool fluid from a desired number of containers.

In the embodiment of FIG. 16, the first tubing 122 includes a peristaltic portion 123, which is configured to engage a peristaltic pump head. In some embodiments, the peristaltic portion may be coated or formed of a wear resistant material to improve durability under the wear conditions of rotary peristaltic pumps. Of course, in other embodiments, the first tubing 122 may be uniformly formed.

FIG. 17 is a schematic of one embodiment of an infusion set 400 which may be employed with an infusion system of exemplary embodiments described herein. According to the embodiment of FIG. 17, the infusion set includes a fluid inlet 404, infusion set tubing 402, and needle 406. The fluid inlet of FIG. 17 is configured as a male luer lock configured to engage the fluid outlet shown in FIG. 16. In the embodiment of FIG. 17, the needle 406 is configured as a single butterfly needle suitable for single site infusion. Of course, any suitable infusion set may be used, including infusions sets having one, two, three, four, or five needles to distribute an infused medicinal fluid, as the present disclosure is not so limited.

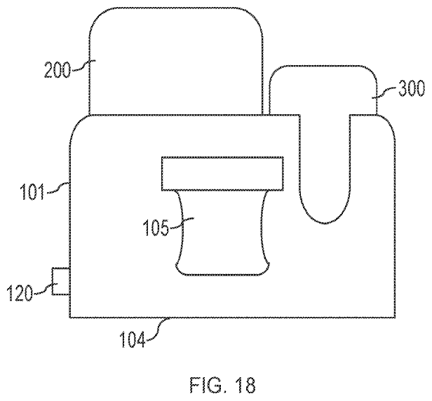

FIG. 18 is a side schematic of another embodiment of an infusion system including a clip 105 configured to allow a pump unit 100, motor unit 200, and/or container 300 to be worn on clothing. For example, the embodiment shown in FIG. 18, the clip is configured as a belt clip formed on a pump unit housing 101 which releasably attaches to a user's belt. Of course, any suitable arrangement may be employed to allow a patient to wear the pump unit, motor unit, and/or container 300, as the present disclosure is not so limited.

While the present teachings have been described in conjunction with various embodiments and examples, it is not intended that the present teachings be limited to such embodiments or examples. On the contrary, the present teachings encompass various alternatives, modifications, and equivalents, as will be appreciated by those of skill in the art. Accordingly, the foregoing description and drawings are by way of example only.

1. A pump unit for an infusion system, comprising:
a housing;
a cavity formed in the housing and configured to receive and support a container, wherein the cavity includes a cavity bottom and a cavity wall;
a spike disposed in the cavity and extending perpendicular to the cavity bottom configured to pierce the container when the container is received in the cavity;
a peristaltic pump head disposed in the housing;
a fluid outlet; and
tubing fluidly coupling the spike to the fluid outlet, wherein the peristaltic pump head is in contact with at least a portion of the tubing,
wherein the housing circumscribes the cavity, spike, peristaltic pump head, and tubing.

2-19. (canceled)

20. A pump unit cartridge, comprising:
a housing;
a cavity formed in the housing including a cavity bottom, and a cavity wall;
a container disposed in the cavity and including an internal cavity and a stopper, wherein the container is retained in the cavity and at least partially surrounded by the cavity wall;
at least one blocking projection that inhibits the container from moving toward the cavity bottom;
a spike disposed in the cavity and extending perpendicular to the cavity bottom configured to pierce the container when the container is moved toward the cavity bottom;
a peristaltic pump head disposed in the housing;
a fluid outlet; and
tubing fluidly coupling the spike to the fluid outlet, wherein the peristaltic pump head is in contact with at least a portion of the tubing.

21. The pump unit of claim 20, wherein the at least one blocking projection is formed on the cavity.

22. The pump unit of claim 20, wherein the at least one blocking projection is formed on the container.

23. The pump unit of claim 20, wherein the at least one blocking projection is disposed between the container and the spike.

24. The pump unit of claim 23, wherein the at least one blocking projection is removable from the pump unit cartridge to allow the container to be moved toward the cavity bottom.

25. The pump unit of claim 24, wherein the at least one blocking projection is configured as a plate which is slidably disposed in a slot formed in the cavity wall.

26. The pump unit of claim 25, wherein the plate is removable from the slot in a direction parallel to a plane defined by the cavity bottom.

27. The pump unit of claim 20, wherein the at least one blocking projection is disposed between the container and the cavity wall.

28. The pump unit of claim 27, wherein the at least one blocking projection is frangible, and wherein the at least one blocking projection is configured to resist movement of the container toward the cavity bottom until a threshold force is applied to the container in a direction of the cavity bottom and the at least one blocking projection breaks.

29. The pump unit of claim 28, wherein the at least one blocking projection is at least three blocking projections.

30. The pump unit of claim 27, wherein the at least one blocking projection is under compression between the container and the cavity wall to generate a frictional force between the container and the cavity wall, wherein the generated frictional force inhibits movement of the container toward the cavity bottom until a threshold force is applied to the container in a direction of the cavity bottom.

31. The pump unit of claim 30, wherein the at least one blocking projection is configured as an O-ring which engages a perimeter of the container.

32. The pump unit of claim 30, wherein the at least one blocking projection is formed of rubber.

33. An infusion system comprising:
a pump unit comprising:
a pump unit housing,
a first cavity formed in the pump unit housing and configured to receive and support a container, wherein the first cavity includes a cavity bottom and a cavity wall,
a first spike disposed in the cavity and extending perpendicular to the cavity bottom configured to pierce the container when the container is received in the cavity,
a motor unit receptacle formed in the pump unit housing;
a peristaltic pump head disposed in the housing;
a fluid outlet, and
tubing fluidly coupling the first spike to the fluid outlet, wherein the peristaltic pump head is in contact with at least a portion of the tubing; and
a motor unit disposed in the motor unit receptacle, comprising:
a battery, and
a motor electrically connected to the battery and having an output shaft directly coupled to the peristaltic pump head,
wherein the motor unit is removable from the motor unit receptacle.

34-36. (canceled)

37. The infusion system of claim 33, further comprising a container disposed in the first cavity fluidly coupled to the first spike.

38. The infusion system of claim 33, wherein the motor unit receptacle includes at least one latch configured to retain the motor unit in the motor unit receptacle.

39. The infusion system of claim 33, wherein the pump unit further comprises a second cavity formed in the housing and a second spike disposed in the second cavity, wherein the second spike is configured to pierce a second container when the second container is received in the second cavity.

40. The infusion system of claim 39, wherein the first spike and the second spike are fluidly coupled to one another to pool fluid from the first container and second container.

41. The infusion system of claim 33, wherein the pump unit housing comprises a clip configured to releasably attach the pump unit to clothing.

42-43. (canceled)
